(19) United States
(12) Patent Application Publication
(10) Pub. No.: US 2008/0058757 A1
(43) Pub. Date: Mar. 6, 2008

(54) Catheter Set for Epidural or Peripheral Nerve Blockade

(75) Inventors: Heinrich Pajunk, Geisingen (DE); Horst Pajunk, Geisingen (DE); Ban C.H. Tsui, Edmonton (CA)

Correspondence Address: AKERMAN SENTERFITT, P.O. BOX 3188, WEST PALM BEACH, FL, 33402-3188, US

(73) Assignee: Pajunk GmbH & Co. KG Besitzverwaltung, Geisingen (DE)

(21) Appl. No.: 11/742,480

(22) Filed: Apr. 30, 2007

(30) Foreign Application Priority Data
Apr. 28, 2006 (DE) DE 102006020363.1

(51) Int. Cl. A61M 31/00; A61B 17/34; A61M 5/178

(52) U.S. Cl. 604/508; 604/21; 607/116

(57) ABSTRACT

A catheter set for the epidural or peripheral nerve blockade, in which a flexible catheter (20) includes a stimulation tip (22) for the localization and positioning of the catheter tip by electro-stimulation. A reinforcing stylet (40) is introducible in the catheter (20). The axial position of the reinforcing stylet (40) in the catheter (20) is so selected that the distal end area (b) of the catheter (20) is un-reinforced and remains flexible.

### FIELD OF INVENTION

The invention concerns a catheter set for epidural or peripheral nerve blockade.

### BACKGROUND OF THE INVENTION

In an epidural nerve blockade an anesthetic is injected into the epidural tissue in order to anesthetize the deeper-lying body parts of the patient. For this, a cannula is stuck into the vertebral canal, and a catheter is introduced via this cannula and is advanced in the epidural space until reaching the area in which the anesthetic is to be applied. In particular, in a thoracic blockade the catheter can be introduced into the sacral or lumbar vertebral segment in order to push the catheter a relatively long length in to the thoracic vertebrae region.

In a peripheral nerve blockade, which is used in particular for the operational or analgetic treatment of the extremities, a cannula is stuck in to the nerve sheath and the catheter is advanced through the cannula in the nerve sheath along the nerve into the region in which the anesthetic is to be applied.

From DE10100976C2 a catheter for nerve blockade is known, which can be positioned with the aid of electro-stimulation. For this, the flexible electric insulating plastic catheter has on its distal end a bare and uninsulated electrically conductive stimulation tip, which is connected via an electrically conductive connection running through the inside of the catheter to a stimulation connector provided at the proximal end of the catheter. A stimulation device connected to the stimulation connector sends electrical stimulation pulses to the distal stimulation tip, in order to stimulate the nerve in the area of this stimulation tip and thereby to provide indication to the anesthetist regarding the position of the distal catheter tip. The catheter must, on the one hand, exhibit a sufficient flexibility in order to be able to flex during advancement through the nerve structure, so that no damage occurs to the nerve. On the other hand, such flexibility can be disadvantageous when the catheter must be advanced over a great distance. In extreme situations, it is even possible that the flexible distal end of the catheter, upon reaching an area of resistance, is bent over backwards.

From U.S. Pat. No. 5,976,110A, a catheter set for peripheral nerve blockade is known, in which the cannula for the insertion in the nerve sheath has a distal stimulation tip, with which the exact position of the cannula tip in the nerve sheath can be determined. A flexible catheter is introduced in this cannula so far that its distal tip corresponds to the distal stimulation tip of the cannula. If the cannula is withdrawn, then the distal tip of the catheter is located in the position previously determined by the stimulation cannula. The catheter can be fed coaxially through a hose adapter that can be seated upon the proximal end of the cannula. The distal end of the catheter can only be positioned in registry with the distal end of the cannula, an advancing of the catheter beyond the distal end of the cannula, as is necessary in particular for an epidural anesthesia, is not provided for.

From EP1002500A1, a catheter set for plexus anesthesia is known, in which with the aid of a stimulation cannula a plastic semi-permanent cannula is introduced in the nerve sheath, through which a catheter can be introduced. In the flexible plastic catheter there is provided a stimulation wire, which is exposed on the distal end of the catheter via openings in the catheter, and is connected on its proximal end to a stimulator. The stimulation wire causes a stiffening of the flexible plastic catheter. The stimulation wire must be continuously located at the distal tip of the catheter during advancement thereof, since the stimulation wire must bring about stimulation of the nerve for positioning at this distal tip. Due to this stimulation wire, the catheter loses its flexibility, which flexibility is desired for the risk-free advancing of the catheter tip.

### SUMMARY

The invention is concerned with a task of providing a catheter set for the epidural or peripheral nerve blockade, which combines an exact positioning of the distal catheter tip and an advancement of the catheter, with as little risk as possible, over a long distance.

The inventive catheter set includes a cannula which serves for sticking in the epidural space or, as the case may be, through the peripheral nerve sheath. This cannula can also be useful in conventional manner in the form of a unipolar stimulation cannula. One such stimulation cannula is preferably in the form of a steel cannula, which is externally coated with an electrically insulating coating, so that only its distal tip remains free in a punctiform area. The exact position of this distal cannula tip in the epidural space, or as the case may be, the peripheral nerve sheath can be localized via a proximal stimulation connector. The distal tip of the cannula can likewise in known manner be in the form of a facett cut or a Tuohy tip or in the form of a sprotte tip.

A catheter is introduced through the cannula into the epidural space, or as the case may be, the nerve sheath, which is a flexible non-conductive catheter, preferably of plastic. The catheter can be entirely in the form of a plastic hose. Catheters can be employed, in which a healical-wound thin wire is introduced in the plastic hose, in order to improve the bend-resistance of the hose, without detracting from the flexibility thereof. The catheter serves for supplying the liquid anesthetic, which is introduced in the proximal end of the catheter via a syringe connector and emerges through at least one exit opening at the distal end of the catheter. In order to be able to precisely position the end of the catheter with its exit opening, the catheter is a stimulation catheter. For this, the catheter exhibits on its distal end an exposed electrical conductive stimulation tip which is connected, via an electrically conductive connection running through the inside of the catheter, for example a thin wire, with an electrical stimulation connector provided at the proximal end of the catheter. The conductive connection is so designed that it keeps free or open to the greatest extent possible the inner lumen of the catheter. If the conductive connection is in the form of, for example, a wire, then the diameter of this wire is substantially smaller than the free inner diameter of the catheter. The exit opening at the distal end of the catheter can run axially through the distal end phase of the catheter, so that the supply of liquid is ejected forwards in the direction of advancement of the catheter. Such a direction of emission is preferred for example in the case of a peripheral nerve blockade. The exit opening can also be provided at the side in the catheter wall on the distal end of the catheter. A radial extrusion of the liquid essentially perpendicular to the axis of the catheter results. Such an emission direction is preferred in part for an epidural nerve blockade. It is also possible to provide two or more exit openings, wherein one axial and one or more radial exit openings can be combined. A stimulation catheter of this type is essentially known from DE10100976C2.

In accordance with invention a re-enforcing stylet is inserted in this flexible catheter, which then serves for stiffening of the flexible catheter and not for electrical stimulation. The stiffening or reinforcing stylet can be pushed up to the distal tip of the catheter, in order to stiffen the catheter up to its distal tip. Thereby an advancement of the catheter is facilitated even over greater distances. The stiffening stylet is variably pulled back from the tip of the catheter during the advancement of the catheter, so that ahead of the distal tip of the reinforcing stylet a flaccid or un-reinforced flexible end area of the catheter remains. This flexible distal tip area of the catheter allows a yielding of flexing of the catheter tip during the advancement upon encountering an area of resistance so that a damaging in particular of nerve tissue or a dura perforation through the catheter tip during advancement is substantially precluded. By the variable axial advancement of the reinforcing stylet in the catheter, the length of the freely-yielding flexible catheter tip can be selected during advancement to conform to the anatomical conditions encountered.

The catheter set thereby unites an optimal stiffness of the catheter over its near complete length for the advancement, with a flexibility of the distal tip of the catheter for avoidance of damage.

In one advantageous embodiment, the catheter has on its proximal end a connection adapter, which has on its side a stimulation connector for the connection of the electrical stimulator and allows the coaxial introduction of the reinforcing stylet in the proximal end of the catheter.

It is further useful to provide a hose adapter at the proximal end of the catheter which makes possible the connection of a syringe to the catheter via a side insertable syringe hose while the reinforcing stylet is introducible coaxially also through the hose adapter. The hose adapter is in particular equipped with a haemostatic valve. Via a syringe hose a fluid can be injected, in particular during the introduction of the catheter. This could be an anesthetic. In particular, however, also a fluid could be injected which widens or dilates the nerve canal or, as the case may be, the epidural space and which facilitates a sliding of the catheter tip during advancement. One such fluid is for example a 5% solution of dextrose in water. This fluid has the dilating as well as buoying or hydroplaning function, without influencing the electrical stimulation of the stimulation tip. The dilation by the liquid can also be used for a supplemental localization of the catheter tip by means of ultrasound. It is also possible to squirt in a physiological salt solution as the liquid, taking advantage of the conductivity of the salt solution to support the electrical nerve stimulation through the catheter tip. The haemostatic valve of the hose adapter makes possible also an aspiration by means of the syringe, in order to check the position of the catheter tip, and for example, to avoid a perforation of the blood vessel.

Preferably the distal tip of the catheter is closed up to the exit opening provided in certain cases axially oriented in the catheter tip. Thereby, it is supplementally insured that the reinforcing stylet cannot exit distally out of the catheter.

### BRIEF DESCRIPTION OF THE DRAWINGS

In the following the invention will be described in greater detail on the basis of the illustrative embodiments shown in the figures. There is shown in:

FIG. 1 a cannula for the catheter set,

FIG. 2 the catheter set according to the invention; and

FIG. 3 an axial section through the distal end of the catheter in enlarged representation.

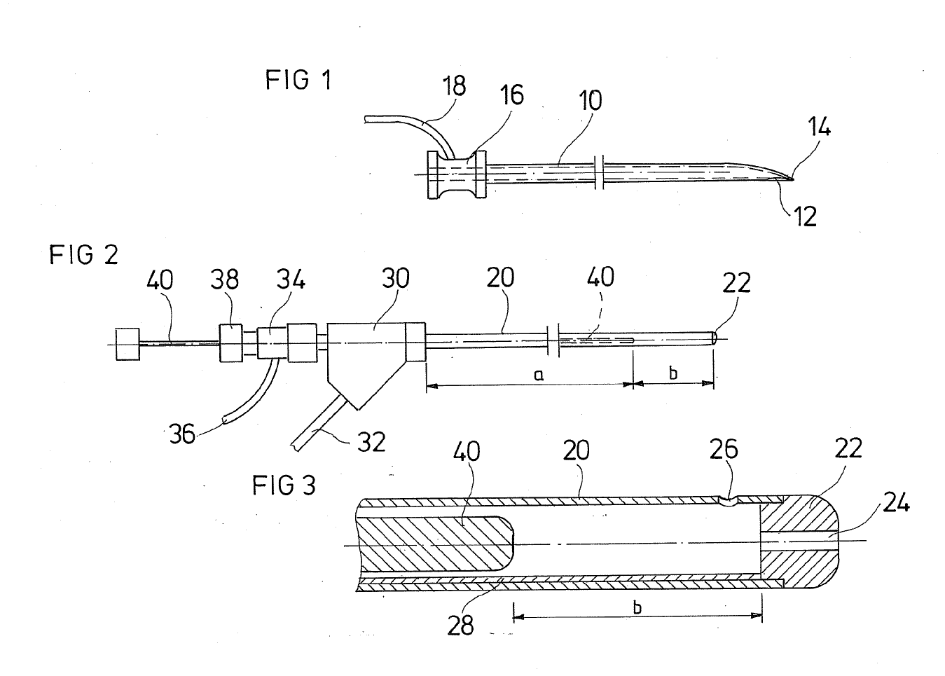

The full set is comprised of the cannula 10 shown in FIG. 1 and the actual catheter set shown in FIG. 2.

### DETAILED DESCRIPTION OF THE PREFERRED EMBODIMENTS

The cannula 10 is preferably a unipolar stimulation cannula comprised of a rigid steel tube, which on its distal end in the illustrated example is in the form of a Tuohy tip 12. Of course, the tip could also be in the form of a facet cut or a Sprotte tip. The cannula 10 is covered over its entire length with an external insulating coating, keeping free essentially only a punctiform tip 14. On the proximal end of the cannula 10 a plastic connector 16 is provided, through which a connection wire 18 is introduced, which contacts the metallic cannula tube and serves for the connection to an electrical stimulator. The catheter, described in greater detail below, can be inserted through the connector 16 into the tube of the cannula 10.

The catheter 20 represented in FIGS. 2 and 3 is a flexible catheter of a nonconductive soft plastic, of which the length can be between 15 cm and 100 cm depending upon application. In certain cases, the wall of the catheter 20 can also be reinforced by an inserted or imbedded wire coil in order to inhibit a kinking or flattening of the catheter 20. The flexible bendability of the catheter is not inhibited thereby. The catheter 20 exhibits on its distal end a stimulation tip 22, which in the shown embodiment is an electrically conductive cap, which is introduced on the distal end of the catheter 20. On the distal end of the catheter 20 at least one exit opening is provided for the liquid supplied through the catheter. In the shown illustrative embodiment an exit opening 24 is a coaxial opening, which extends through the stimulation tip 22. A further exit opening 26 is provided directly behind the stimulation tip 22 in the wall of the catheter 20. The exit openings 24 and 26 can be provided alternatively or collectively. An electrically conductive connection extends along the catheter 20, which in the illustrative embodiment is in the form of a thin wire 28. This wire 28 is conductively connected distally with the stimulation tip 22 and extends the entire length of the catheter 20 to the proximal end thereof. On the proximal end of the catheter 20 a connection adapter 30 of plastic is provided. The connection adapter 30 receives a stimulation connector 32 which is introduced in the connection adapter 30 as a cord or flexible wire and, upon securing the catheter 20 in the connection adapter 30, contacts the wire 28. The stimulation connection 32 serves for connection of the electrical stimulator. Coaxially in the proximal end of the connection adapter 30 a hose adapter 34 is seatable. An infusion or syringe hose 36 leads sideways into the hose adapter 34. On the syringe hose 36 a not shown syringe can be connected. Via the injection hose 36 and the hose adapter 34 the liquid can be supplied in to the catheter 20 or a liquid can be aspirated via the catheter 20. For this the hose adapter 34 exhibits a haemostatic valve. The hose adapter 34 can be connected on its proximal end by a fastener or lock 38, in order to make possible an injection or aspiration via the hose adapter 34 and the injection hose 36. The connection adapter 30 and the hose adapter 34 form an axially continuous channel communicating axially with catheter 20. A reinforcing stylet 40 can be inserted through this channel from the proximal end through the hose adapter 34 and the connection adapter 30 into the catheter 20. The length of the reinforcing stylet 40 is so determined that this, when it is completely inserted in the catheter 20, reaches with its distal end up to the inner distal end of the catheter 20; in the illustrated embodiment, to the inner end of the stimulation cap 22. The reinforcing stylet 40 is a stiff steel wire, which is elastically flexible, however kink resistant. The diameter of the reinforcing stylet 40 is somewhat smaller than the inner diameter of the catheter 20, so between the outer jacket surface of the reinforcing stylet 40 and the inner jacket surface of the catheter 20 a ring-gap remains, through which a liquid supplied through the injection hose 36 can pass to the distal catheter tip.

The reinforcing stylet 40 can be advanced distally a selectable distance in the catheter 20. In the axial longitudinal area of the a catheter, in which the reinforcing stylet 40 is located, the soft-flexible catheter 20 is reinforced by the reinforcing stylet 40. In the distal end area d of the catheter 20, in to which the reinforcing stylet 40 is not pushed, the catheter 20 maintains its soft flexible characteristic and can yield to obstructions without resistance. By axial displacement of the reinforcing stylet 40 the length of this soft tip area b of the catheter 20 can be adapted to the anatomic conditions, through which the catheter tip must respectively actually be pushed.

The process of epidural or peripherable nerve blockade occurs in the following steps:

First, the cannula 10 punctures the vertebral canal and the epidural (in the case of epidural nerve blockade) or the nerve sheath (in the case of peripheral nerve blockade). Via the connection wire 18 the cannula 10 is connected to an electrical stimulator which delivers the stimulation pulse via the tip 14 to the nerve tissue adjacent the tip 14. Through these nerve stimulations the exact position of the cannula tip 12 can be located and positioned.

After the cannula 10 is positioned with its tip 12, the catheter 20 is introduced through the connector 16 into the cannula 10. The catheter 20 exits with its distal tip from the distal end of the cannula 10 and is pushed into the epidural space or, as the case may be, the nerve sheath. In the catheter 20 at this time the reinforcing stylet 40 is introduced.

During the advancement of the catheter 20 the stimulation connection 32 is connected to the electrical stimulator and stimulation pulses are conducted via the stimulation connection 32 and the wire 28 to the stimulation tip 22 so that the respective position of the distal tip of the catheter 20 can be localized via the nerve stimulation by means of the stimulation tip 22. Depending upon the anatomic condition in this area, through which the distal tip of the catheter 20 is actually being pushed, the reinforcing stylet 40 can be more or less advanced axially towards the front in the catheter 20. Thereby the anesthetist can influence the stiffness or, as the case may be, the yieldability of the distal tip of the catheter 20 and optimally adapt to the anatomic conditions.

During the advancement of the catheter 20 via the injection hose 36 a liquid can be introduced into the catheter 20, which exits via the exit openings 24 or as the case may be 26. This liquid can serve to dilate the space through which the distal tip of the catheter 20 is actually being pushed and to form a liquid padding or slip layer, which facilitates the advancement of the catheter tip. For this, for example, an aqueous dextrose solution can be employed. Alternatively a salt solution can be injected, which on the basis of its electrical conductivity can, when desired, improve the contact between the stimulation tip 22 and the surrounding nerve tissue. As soon as the distal end of the catheter 20 is position in the desired position by means of the stimulation tip 22, the reinforcing stylet 40 is withdrawn out of the catheter 20 and the connection adapter 30 is disconnected from the catheter 20. Now the cannula 10 can be withdrawn proximally from the catheter 20 and the catheter 20 can remain in place for continuous anesthesia, or for a later follow-up dosing of the anesthetic. With the aid of the stimulation tip 22 the position of the catheter 20 can also be subsequently corrected as desired.

### REFERENCE NUMBER LIST

10 Cannula
12 Tuohy tip
14 Tip
16 Connector
18 Connection wire
20 Catheter
22 Stimulation tip
24 Exit opening
26 Exit opening
28 Wire
30 Connection adapter
32 Stimulation connection
34 Hose adapter
36 Injection hose
38 Seal
40 Reinforcing stylet

We claim:

1. A catheter set for an epidural or peripheral nerve blockade, comprising:
a cannula (10);
a flexible catheter (20) of a soft electrically insulating plastic introducible through the cannula (10);
at least one exit opening (24, 26) for a liquid provided at a distal end of the catheter (20);
an electrically conductively exposed stimulation tip (22) at the distal end of the catheter (20);
an electrical stimulation connection (32) provided at a proximal end of the catheter (20); and
a conductive connection (28) extending through and inside of the catheter (20) connecting the stimulation tip (22) with the stimulation connection (32),
wherein the catheter (20) is provided with a reinforcing stylet (40), which is positionable in its axial position in the catheter (20) in such a manner that ahead of a distal end of the reinforcing stylet (40) a flexible end area (b) of distal selectable length of the catheter (20) remains.

2. The catheter set according to claim 1, wherein at the proximal end of the catheter (20) a connection adapter (30) is provided, which receives the stimulation connection (32) and through which the reinforcing stylet (40) is coaxially introducible.

3. The catheter set according to claim 2, wherein a hose adapter (34) is seatable proximally on the connection adapter (30), which has a haemostatic valve and communicates with an injection hose (36).

4. The catheter set according to claim 3, wherein the reinforcing stylet or mandrell (40) is introducible coaxially through a closable proximal end of the hose adapter (34).

5. The catheter set according to claim 1, wherein the distal end of the catheter (20) is closed at least up to the exit opening (24).

6. The catheter set according to claim 1, wherein the cannula (10) is a unipolar stimulation cannula.

7. A process for epidural or peripheral nerve blockade including the following steps:
inserting a rigid cannula in an epidural space or in a peripheral nerve sheath;
introducing a flexible catheter through the cannula into the epidural space or the peripheral nerve sheath;
localizing with electrical stimulation the actual position of the distal tip of the catheter;
introducing a reinforcing stylet into to the catheter;
variably selecting the axial position of the reinforcing stylet in the flexible during advancement of the catheter such that ahead of the distal end of the reinforcing stylet a flexible distal end area of the catheter of appropriate length remains in accordance with the anatomic conditions at the position of the catheter tip.

8. The process according to claim 7, wherein a liquid is supplied via the injection hose during the advancement of the catheter, which is emitted at the distal end of the catheter and which dilates the path of advancement of the distal catheter tip.

9. The process according to claim 7, wherein during the advancement of the catheter an electrically conductive liquid is supplied via an injection hose, which is emitted at the distal tip of the catheter and improves the electrical conductivity between the stimulation tip of the catheter and the surrounding nerve tissue.
